(19) United States
(12) Patent Application Publication
(10) Pub. No.: US 2009/0062670 A1
(43) Pub. Date: Mar. 5, 2009

(54) HEART MONITORING BODY PATCH AND SYSTEM

(76) Inventors: Gary James Sterling, Westlake Village, CA (US); James Mazeika, Thousand Oaks, CA (US); William Rissmann, Deephaven, MN (US)

Correspondence Address: IPLA P.A., 3580 WILSHIRE BLVD., 17TH FLOOR, LOS ANGELES, CA, 90010, US

(21) Appl. No.: 12/122,562

(22) Filed: May 16, 2008

Related U.S. Application Data
(60) Provisional application No. 60/969,113, filed on Aug. 30, 2007.

(51) Int. Cl. A61B 5/0402; G06Q 50/00

(52) U.S. Cl. 600/509; 705/2

(57) ABSTRACT

Provided is a diagnostic patch system for monitoring and storing patient information, which includes sensors, a data storage unit, and a transceiver. Each of the sensors is attached to a skin to detect a patient data. The data storage unit is configured to store stream of the detected patient data from the plurality of sensors. The transceiver, connected with the sensors and the data storage unit, communicates the stream of the patient data with an analyzer, and the analyzer is configured to process and analyze the stream of the patient data. Two or more diagnostic patch systems can communicate with each other.

### CROSS REFERENCES

This is a non-provisional application claiming priority of Provisional Application No. 60/969,113 for “A disposable patch that monitors and stores heart rate, oxygen and patient activity information for 24/48 hours” filed Aug. 30, 2007.

### BACKGROUND OF THE INVENTION

This invention relates to a diagnostic body patch system for monitoring cardiovascular activities of a patient.

Medical, medicinal and athletic sciences have been constantly driven to track biological or physiological state variables of a human body which interacts ceaselessly with the environment in terms of a function of time. In order to make a correct assessment of the bodily state variables, the bodily condition under which the body functions need to be monitored for preventive and diagnostic purposes. Ongoing research in medical and sports science further encourages monitoring a patient's body and its environmental parameters to thus analyze correlation of those parameters for therapeutic purposes. Applicants have searched and reviewed conventional arts encompassing a bodily patch and its applied variations, which have yet to meet the market demands.

Stivoric et al., U.S. Pat. No. 7,020,508 discloses a detecting apparatus that includes a housing support section(s), a housing removably attached thereto, one or more sensors and a processor. An alternate apparatus measures heat flux and includes a known resistivity base member, a processing unit and two temperature measuring devices, one in thermal communication with the body through a thermal energy communicator and the other in thermal communication with the ambient environment. A further alternate apparatus includes a housing or flexible section having an adhesive material on a surface thereof for removably attaching the apparatus to the body. A further alternate apparatus includes a housing having an inner surface having a concave shape in a first direction and convex shape in a second direction substantially perpendicular thereto. Also, an apparatus for detecting heart related parameters includes one or more filtering sensors for generating filtering signals related to the non-heart related motion of the body. The sensor device generates data indicative of various physiological parameters of an individual such as the individual's heart rate, pulse rate, beat-to-beat heart variability, EKG or ECG, respiration rate, skin temperature, core body temperature, heat flow off the body, galvanic skin response or GSR, EMG, EEG, EOG, blood pressure, body fat, hydration level, activity level, oxygen consumption, glucose or blood sugar level, body position, pressure on muscles or bones, and UV radiation exposure and absorption.

Barton et al., U.S. Pat. No. 6,814,706 discloses a skin patch includes first and second layers of material and a telesensor sandwiched between the first and second layers. The first layer has a coating of skin compatible adhesive material on its face that is remote from the second layer. Although the invention is described with reference to a temperature sensor, the invention is also applicable to other telesensors, for example telesensors that emit signals representative of heart rate, heart rate interbeat interval activity level, including activity level at the sensor location, and blood oxygen level.

Fadem et al., U.S. Publication No. 2005/0280531 discloses a wireless biopotential monitoring system composed of a wireless electrode module which can be attached to a disposable electrode strip. Such a device can be affixed to a patient's skin and will transmit the physiological signals to a remote receiver where the signals can be monitored by a clinician. The device is powered by a fuel-air battery. The device would remain packaged in an airtight package until it needs to be applied at which time either the wounded soldier would apply the device himself/herself or it would be applied by another soldier or corpsman. The device would begin to measure brainwave activity, heart rate, and dissolved oxygen level. The device would also identify the wounded soldier's location using the onboard GPS receiver. The physiologic data along with the soldier's position would then be transmitted to a remote receiver.

Lawson et al., U.S. Pat. No. 7,129,836 discloses a data acquisition system can include an acquisition device and a receiving device. The acquisition device includes inputs that receive data from sensors connected to a subject, a wireless and/or a wired transmitter that transmits data received by the inputs, and a housing carrying at least some of the components of the acquisition device. The housing may be wearable by a patient. The acquisition device may be switchable between a tethered data transmission mode and an untethered data transmission mode. The receiving device includes a receiver that receives data transmitted by the acquisition device, and may include an output that outputs data to a host. The system may be configured to transmit data from the data acquisition device to the local monitor point-to-point. Some examples of patient sensors that can be used include electrocardiograph (ECG) electrodes, non-invasive blood pressure (NBP) cuffs, pulse oximetry probes, temperature probes, cardiac output probes, and invasive blood pressure transducers.

Nikolic et al., U.S. Pat. No. 6,436,052 discloses systems and methods for the determination of an individual's rate of oxygen consumption, in order to determine the amount of work that is performed by the individual's body. A heart monitor measures the heart rate of the individual and an accelerometer measures the acceleration of the body. The heart rate and acceleration outputs are stored locally on a storage device. The outputs can be downloaded to a local base station, that in turn transmits the outputs to a central clearinghouse. The clearinghouse receives and stores the output on a central mass storage device. At the clearinghouse the raw data is processed into a usable form and the rate of oxygen consumption is mathematically determined in order to determine the amount of work that is performed on the individual's body. The processing includes separating the static and dynamic acceleration components, calculating the dynamic acceleration magnitude, calculating the maximum change in acceleration. filtering the dynamic acceleration component. and graphing the resulting filtered dynamic acceleration with respect to time.

Welch et al., U.S. Publication No. 2006/0238333 discloses a method for performing context management, said method comprising the steps of: producing a continuous physiologic signal, as detected by a monitoring device; associating at least one unique hardware identifier to said continuous physiologic signal and binding a unique patient identifier to said continuous signal wherein a change in said physiologic signal in which said signal is no longer continuous will cause the unique patient identifier to unbind from said signal.

Kilot et al, U.S. Publication No. 2006/0100530 discloses methods and systems for long term monitoring of one or more physiological parameters such as respiration, heart rate, body temperature, electrical heart activity, blood oxygenation, blood flow velocity, blood pressure, intracranial pressure, the presence of emboli in the blood stream and electrical brain activity are provided. Data is acquired non-invasively using ambulatory data acquisition techniques.

Wehman et al., U.S. Publication No. 2005/0054938 discloses a method and calculations to determine an individual's, or several individuals', simultaneous rates of oxygen consumption, maximum rates of oxygen consumption, heart rates, calorie expenditures, and METS (multiples of metabolic resting rate) in order to determine the amounts of work that is performed by the individual's body. A heart monitor measures the heart rate, and an accelerometer measures the acceleration of the body along one or more axes. An altimeter measures change in altitude, a glucose monitor measures glucose in tissue and blood, and thermometers, thermistors, or thermocouples measure body temperature. Data including body fat and blood pressure measurements are stored locally and transferred to a processor for calculation of the rate of physiological energy expenditure. Certain cardiovascular parameters are mathematically determined. Comparison of each axis response to the individual's moment can be used to identify the type of activity performed and the information may be used to accurately calculate total energy expenditure for each physical activity. Energy expenditure may be calculated by assigning a separate proportionality coefficient to each axis and tabulating the resulting filtered dynamic acceleration over time, or by comparison with previously predetermined expenditures for each activity type. A comparison of total energy expenditure from the current activity is compared with expenditure from a previous activity, or with a baseline expenditure rate to assess the level of current expenditure. A measure of the individual's cardiovascular health may be obtained by monitoring the heart's responses to various types of activity and to total energy expended.

Tsoukatis, U.S. Pat. No. 7,161,484 discloses a system for monitoring medical parameters of a being, in particular a human being, comprising medical functional means including at least one sensor section for detecting at least one predetermined medical parameter, a transmitting means for transmitting the medical parameter(s) detected by said sensor section, said transmitting means being adapted to be provided at the being, and a remote serving means for receiving and processing the medical parameter(s) from said transmitting means and providing instructions and/or data on the basis of the processed medical parameters.

Quy, U.S. Pat. No. 7,156,809 discloses a method and apparatus for a wireless health monitoring system for interactively monitoring a disease or health condition of a patient by connecting a mobile phone to or with a digital camera and/or a medical monitoring device. The health related data or visual information from the camera is transmitted to a server using standard internet protocols and may be integrated with various operating systems for handheld or wireless devices, especially those with enhanced capabilities for handing images and visual data.

Parker et al., U.S. Pat. No. 6,997,882 discloses methods and devices for monitoring a subject by acquiring 6-DOF data regarding the subject, and by using that data to obtain information about the subject's movements in three-dimensional space. Information regarding the subject's movements is optionally, combined with information regarding the subject's physiological status so that comprehensive knowledge regarding the subject may be acquired by those monitoring the subject.

Lind et al., U.S. Pat. No. 6,889,165 discloses an intelligent microsensor module (10, 100, 210, 300, 355, 410) that can fuse data streams from a variety of sources and then locally determine the current state of the environment in which the intelligent microsensor is placed. The resultant state rather than raw data is communicated to the outside world when the microsensor is queried. The intelligent microsensor module (10, 100, 210, 300, 355, 410) of the present invention can locally determine and execute an action to be taken based on the determined state of the environment. The module (10, 100, 210, 300, 355, 410) can be readily reconfigured for multiple applications.

Lin et al., U.S. Pat. No. 6,847,294 discloses a radio medical monitoring system, which comprises a modem; a central processing unit (CPU) connected with the modem for digital data transmission therewith; a read-only-memory (ROM) connected with the CPU; a memory connected with the CPU; one or a plurality of digital medical sensors connected with the CPU for transmitting signals from a subject under examination to the CPU: a radio transceiver connected with the modem for receiving/transmitting radio waves and performing an analog signal transmission with the modem. The monitoring system of the present invention has Group ID (Gill) and Sort ID functions, and automatically replies according to the order of the Sort ill after identifying the Group ill and confirming that it is necessary to reply. The present invention also discloses a radio medical monitoring method.

Amano et al., U.S. Pat. No. 6,241,684 discloses a device, which is capable of determining the maximum oxygen uptake quantity without the restriction of a large device or requiring troublesome operations to be carried out. The device displays the upper and lower limit values for the pulse rate corresponding to an appropriate exercise intensity, and realizes in a wireless manner by means of optical communications the sending and receiving of information such as pulse wave signals to and from an information processing device which processes pulse wave information. The device is provided with a pulse wave detector 101 for detecting the test subject s pulse waveform; an FFT processor 103 for determining the test subject s heartbeat rate from the pulse waveform; a body motion detector 104 for detecting body motion when the test subject is running; an FFT processor 106 for determining the pitch from body motion during running by the test subject; exercise intensity calculator 108 for determining pitch, the test subject s stride, and the exercise intensity from body motion during running; and a nomogram recorder 109 for recording the relationship indicated by an Astrand-Ryhming nomogram, and determining the maximum oxygen uptake quantity from the heart rate and exercise intensity. The obtained maximum oxygen uptake quantity is divided by the test subject's body weight, to calculate the maximum oxygen uptake quantity per unit body weight. Next, the maximum oxygen uptake quantity and pulse according to sex are determined, and the pulse rate is multiplied by the upper and lower limit value coefficients, to determine the upper limit value UL and the lower limit value LL for the pulse rate.

Sum et al., U.S. Pat. No. 5,491,474 discloses an invention relating to a telemetric transmitter unit, by means of which signals detected by one electrode or several electrodes connected to the transmitter are wirelessly transmittable to a separate receiver by using a magnetic proximity field. The transmitter electronics (6) is coupled in a fixed manner to each electrode (4) by means of a conductive plastic layer (5). The transmitter electronics (6), the electrodes (4) and the conductive plastic layer (5) are cast and/or jointed together with plastic (1, 2, 3) to form an integrated transmitter unit.

Okuda et al., U.S. Pat. No. D519,636 discloses the ornamental design for a heartbeat detector-transmitter, as shown in the figures of the patent.

### SUMMARY OF THE INVENTION

The present invention is contrived to overcome the conventional disadvantages. An objective of the invention is to provide a diagnostic patch system that is attached to the body of a patient to monitor-and-store patient data.

Another objective is to provide a diagnostic patch system that enables communication of the patient data with an analyzer for analysis and interpretation.

To achieve these and other objectives, a diagnostic patch system according to an aspect of the invention includes a plurality of sensors, a data storage unit, a transceiver, and a flexible support. Each of the plurality of sensors is configured to be attached to one or more skin surface areas to detect a patient data. The data storage unit is configured to store stream of the detected patient data from the plurality of sensors.

The transceiver, connected with the plurality of sensors and the data storage unit, is configured to communicate the stream of the patient data with an analyzer, and the analyzer is configured to process and analyze the stream of the patient data. The flexible support engages thereto the sensors, the data storage unit, and the transceiver, and the flexible support is detachably attached to one or more skin surface areas of a patient. The sensors comprise an electrocardiogram (ECG) sensor having skin surface electrodes where the skin surface electrodes comprise two or more ECG channels.

The plurality of sensors may comprise an electrocardiogram (ECG) sensor. The ECG sensor may comprise three or more skin surface electrodes, and the three or more skin surface electrodes are configured to form two or more ECG channels. At least two ECG channels may be perpendicular to each other. The ECG sensor may be connected with the data storage unit so as to transfer and store output to the data storage unit. The plurality of sensors may comprise a transcutaneous oxygen sensor, and the transcutaneous oxygen sensor may be connected with the data storage unit so as to transfer and store output to the data storage unit.

The plurality of sensors may comprise an accelerometer for detecting kinetic activity of the patient, and the accelerometer may be connected with the data storage unit so as to transfer and store output to the data storage unit. The data storage unit may comprise one or more memory chip.

The diagnostic patch system may further comprise a membrane button configured to insert a marking event in the stream of the detected patient data, an optical indicator for indicating status of the sensors' contact to the skin surface. The optical indicator may comprise one or more light-emitting diodes, or a control circuitry configured to control behavior of the plurality of sensors, the data storage unit, and the transceiver.

The transceiver may be connected with the analyzer through a wired communication or through a wireless communication. Alternatively, the transceiver may be connected with the analyzer through a socket provided in the analyzer.

In an embodiment, the analyzer may comprise a computer. The diagnostic patch system may be disposable. Alternatively, some of the components may be reusable. The transceiver may be configured to communicate with another diagnostic patch system. For a better performance, the diagnostic patch system may further comprise an electromotive force device configured to provide electric power to the sensors, the data storage unit, and the transceiver. The electromotive force device may comprise a battery. The diagnostic patch system may further comprise a flexible support configured to engage the plurality sensors, the data storage unit, and the transceiver so as to fix them in place.

The flexible support may be further configured to attach the diagnostic patch system to the skin of the patient detachably. The flexible support comprises first and second adhesive surface portions. The plurality of first adhesive surface portions configured to engage one of the sensors, the data storage unit, and the transceiver. The plurality of second adhesive surface portions configured to be attached to the skin of the patient. The plurality of second adhesive surface portions comprise skin safe adhesive.

The sensors, the data storage unit, and the transceiver are disposed apart from one another on the flexible support so as to make the diagnostic patch system substantially flexible across the disposed sensors, the data storage unit, and transceiver, and the diagnostic patch system comprises a plurality of flexible electrical wires connecting the sensors, the data storage unit, and the transceiver.

In certain embodiments, each of the first adhesive surface portions may be substantially surrounded by the second adhesive surface portions such that each of the sensors, the data storage unit, and the transceiver is surrounded by the second adhesive surface portions of the flexible support and securely pressed down and contacted to the skin to obtain a good electrical contact.

Further, the first adhesive surface portions of the flexible support are attached to tightly follow top surfaces of the sensors, the data storage unit, and the transceiver such that the second adhesive surface portions are securely attached to the skin. Each of the sensors, the data storage unit, and the transceiver comprises a flat bottom surface such that the first adhesive surface portions meet the second adhesive surface portions with angles larger than 90 degrees at borders between the first and second adhesive surface portions.

Another aspect of the invention provides an analyzer for processing and analyzing the stream of the patient data from a plurality of diagnostic patch systems. The analyzer comprises an interface device, an information processing device, and one or more output devices. The interface device is configured to communicate with each of the plurality of diagnostic patch systems.

The information processing device is configured to analyze and interpret the stream so as to extract a plurality of temporal patient data and to correlate the plurality of temporal patient data. The one or more output devices are configured to present the temporal patient data. The interface device may comprise a socket having electrical terminals and a radio-frequency transceiver. The plurality of temporal patient data may comprise a plurality of ECG data, a temporal change of oxygen consumed by the patient, and a temporal change of kinetic variables. The plurality of temporal patient data may further comprise a cardiovascular data, and a pulmonary data.

The diagnostic patch system has numerous advantages in that: the patch system with sensors and memory can be easily attached to a patient; the patient data can be collected in a wireless mode; and the patient data stored in the memory can be downloaded to an analyzing processor and reviewed over the internet with ease.

Although the present invention is briefly summarized, the full understanding of the invention can be obtained by the following drawings, detailed description, and appended claims.

### BRIEF DESCRIPTION OF THE DRAWINGS

These and other features, aspects and advantages of the present invention will become better understood with reference to the accompanying drawings, wherein:

FIG. 1 is a top plan view illustrating a diagnostic patch system according to an embodiment of the invention;

FIG. 2 is a side plan view of the diagnostic patch system of FIG. 1;

FIG. 3 is a bottom plan view of the diagnostic patch system of FIG. 1;

FIG. 4 is a perspective view of a diagnostic patch system worn on a patient according to an embodiment of the invention;

FIG. 5 is another bottom plan view of the diagnostic patch system of FIG. 1;

FIG. 6 is a block diagram of a circuitry board according to an embodiment of the invention;

FIG. 7 is another top plan view of a diagnostic patch system according to an embodiment of the invention;

FIG. 8 is a schematic diagram illustrating a diagnostic patch system communicating with an analyzer according to an embodiment of the invention;

FIG. 9 is a schematic block diagram illustrating an analyzer communicating with a diagnostic patch system according to an embodiment of the invention;

FIG. 10 is a schematic diagram illustrating two diagnostic patch systems communicating with each other according to an embodiment of the invention;

FIG. 11 is a bottom plan view illustrating a flexible support according to an embodiment of the invention; and

FIG. 12 is a partial cross-sectional view of a diagnostic patch system.

### DETAILED DESCRIPTION OF THE INVENTION

An embodiment of the present invention relates to a disposable patch that monitors and stores heart rate, oxygen and patient activity information for 24/48 hours. The electrode patch contains three skin surface electrodes providing two electrocardiogram (ECG) channels, a transcutaneous oxygen sensor and an accelerometer which are used to gather patient data over a 24/48 hour period from an ambulatory human patient. The electrode patch contains a memory chip used to store the data gathered by the sensors. The electrode patch also contains a transmitter/receiver used to facilitate the transfer of data to an external receiver. The patch will have a membrane button for the patient to push when feeling symptomatic, marking the event. The patch will also have a small light letting the patient know the electrodes have good contact. The monitor will send information to a device that will interpret the gathered information and format the information for the physician to evaluate.

In an embodiment, the system may comprise a probe or diagnostic patch system which holds the sensors and electronics and an analyzer/computer with interpretation software that receives the data, interprets the data, and formats to print the data.

In another embodiment, the diagnostic patch system and the analyzer may communicate with each other for better performance. For example, a physician prescribes the procedure and monitors to evaluate the patient's heart rate, oxygen capacity and correlate the monitored values with the patient's daily activities. The physician places the monitoring patch on the chest area of the patient and translate the patient using the monitored data. The patient returns to the physician after wearing the monitoring patch for the 24 to 48 hour recording period. The physician removes the monitoring patch, downloads the recorded heart rate, oxygen and activity information to an analyzer/computer. The computer immediately analyzes the information and creates a complete report for physician review. The physician reviews the information, makes a diagnosis and prescribes a treatment or therapy for the patient. The physician can print a report for documentation in the patient's file. The physician then bills the patient's insurer for payment.

In still another embodiments of the invention, the patch can be disposable or some components can be reused. Two patches can be built to communicate with each other measuring additional disease parameters like HF lung congestion and respiration. The monitor can be used to evaluate sleep apnea in a sleep clinic. The monitor can be used during pharmaceutical drug studies, evaluating patient's reaction to drugs. The monitor can be also used to evaluate athletic performance.

Within the disposable patch, three skin electrodes may be disposed to collect two channel ECG data, a transcutaneous oxygen sensor to collect oxygen information, a circuit board with an accelerometer to record patient activity, a battery and data storage chip. The patch adheres to the patient's chest by a skin safe adhesive collects the heart rate, oxygen and patient activity information for a period of 24 to 48 hours. The information is then downloaded to a computer to interpret, format, and print the information. This procedure qualifies for payment by government and private insurance plans.

Currently ambulatory (wearable) monitors collect information on a patient's heart rate and life threatening arrhythmias utilizing monitors attached to wires connected to the patient's chest. This new small disposable monitor according to an embodiment of the invention eliminates the external wires, simultaneously collects heart rate, oxygen and patient activity data providing more complete information at one time for patients being evaluated for cardiac and pulmonary problems. This leads to a more complete and accurate diagnosis. Patients do not need to wear multiple monitors or take separate tests. This inexpensive disposable monitor collects data on three parameters at one, then downloads the information to a computer that formats the information for the physician's use. Potential patients includes cardiovascular disease, cardiovascular arrhythmia, sleep apnea, and pulmonary dysfunction patients.

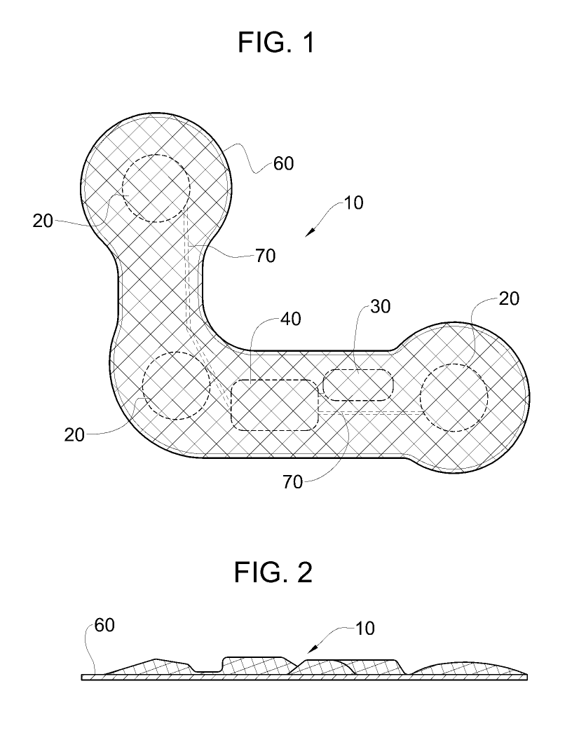

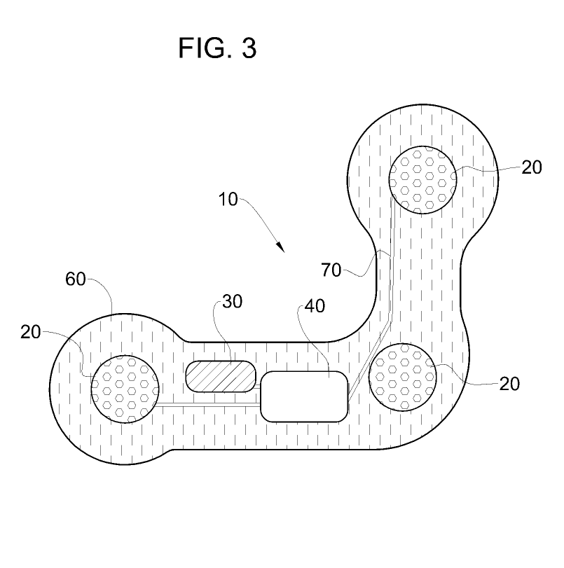

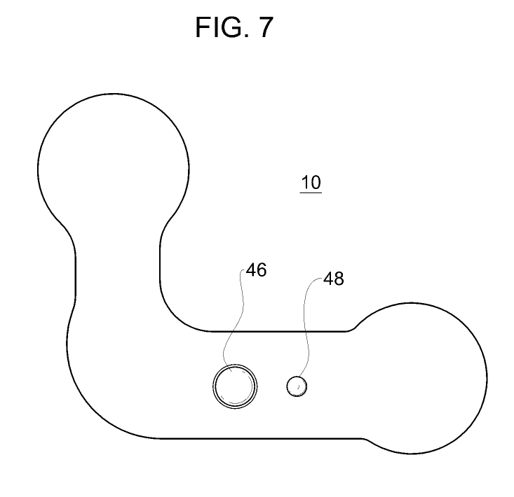

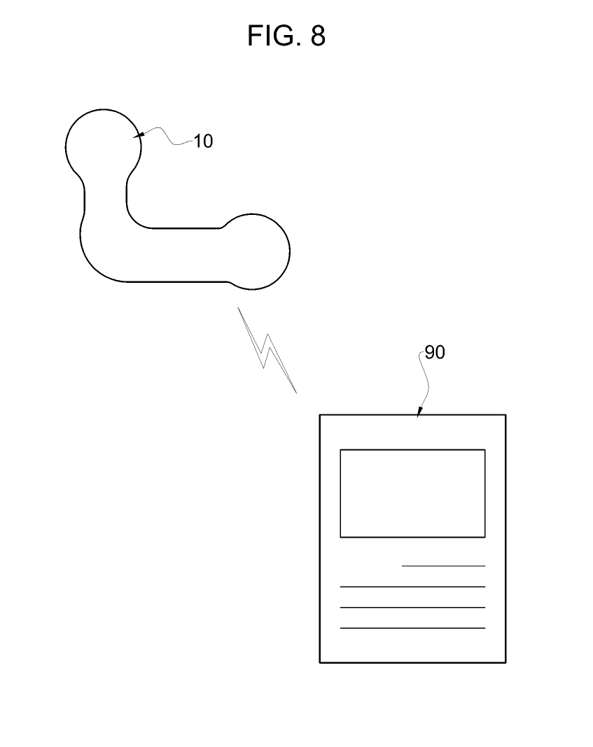

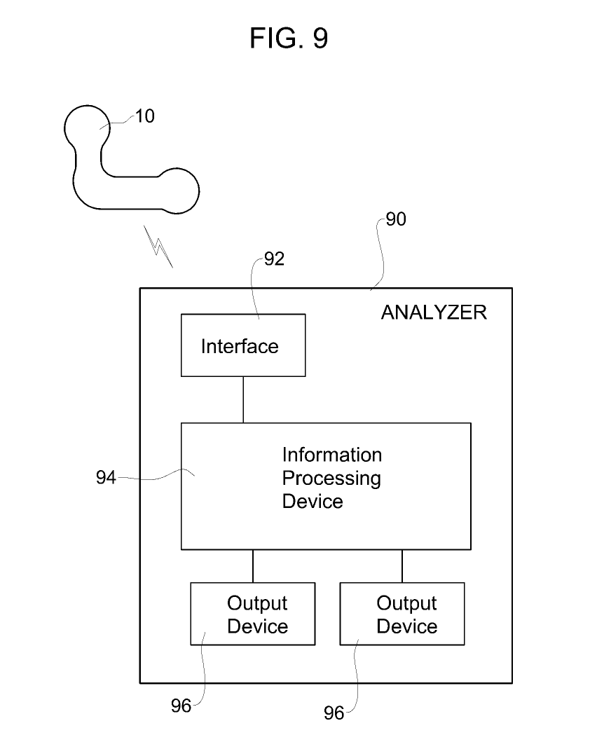

FIGS. 1-7 show a diagnostic patch system 10 according to an embodiment of the invention. FIGS. 8 and 9 show an analyzer 90 communicating with a diagnostic patch system 10. An aspect of the invention provides the diagnostic patch system 10 for monitoring and storing patient information, which comprises a plurality of sensors 20, a data storage unit 43, a transceiver 42, and a flexible support 60. Each of the plurality of sensors 20 is configured to be attached to one or more skin surface areas to detect a patient data.

The data storage unit 43 is configured to store stream of the detected patient data from the plurality of sensors 20.

The transceiver 42, connected with the plurality of sensors 20 and the data storage unit 43, is configured to communicate the stream of the patient data with the analyzer 90, and the analyzer 90 is configured to process and analyze the stream of the patient data as shown in FIGS. 8 and 9.

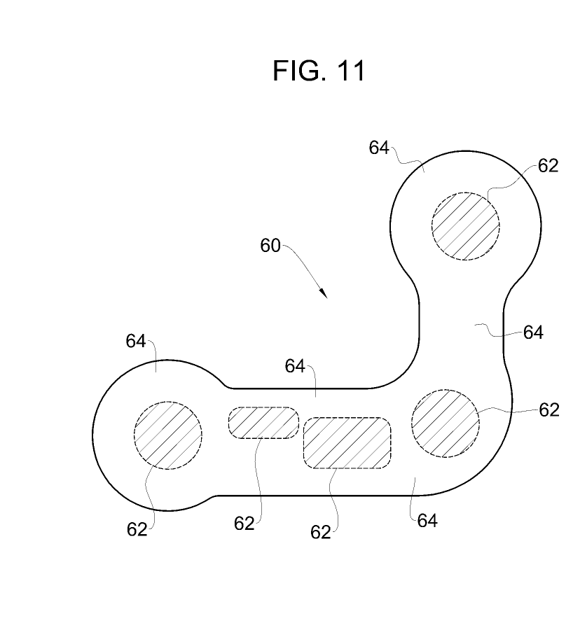

The flexible support 60 engages thereto the sensors 20, the data storage unit 43, and the transceiver 42, and the flexible support 60 is detachably attached to one or more skin surface areas of a patient as shown in FIG. 11.

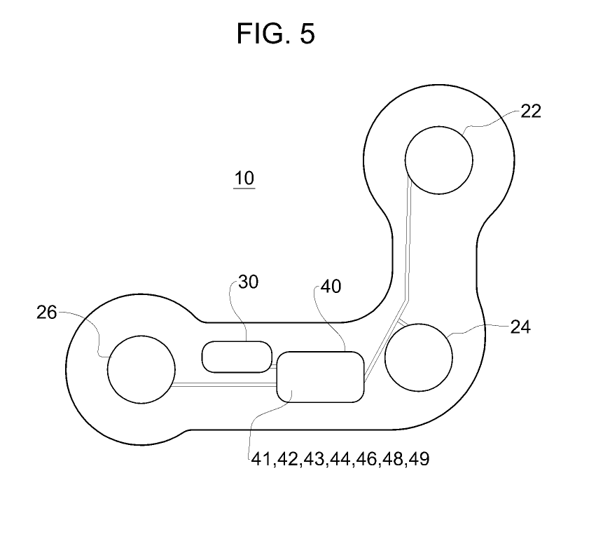

The plurality of sensors 20 may comprise an electrocardiogram (ECG) sensor. The ECG sensor 20 may comprise three or more skin surface electrodes 22, 24, 26 (refer to FIG. 5), and the three skin surface electrodes 22, 24, 26 are configured to form two ECG channels. At least two ECG channels may be perpendicular to each other. In the illustrated embodiment, a first ECG channel may be formed between the skin surface electrodes 22, 24, and a second ECG channel may be formed between the skin surface electrodes 24, 26.

The sensors 20 comprise electrocardiogram (ECG) sensor having skin surface electrodes, and the skin surface electrodes 22, 24, 26 comprise two or more ECG channels. The data storage unit 43 may be configured to store the stream of patient data for twenty four (24) to forty eight (48) hours. The diagnostic patch system 10 may be disposable after use.

The ECG sensor 20 may be connected with the data storage unit 43 so as to transfer and store output from the ECG sensor 20 to the data storage unit 20 as shown in FIG. 5. The plurality of sensors 20 may comprise a transcutaneous oxygen sensor 30, and the transcutaneous oxygen sensor 30 may be connected with the data storage unit 43 so as to transfer and store output from the transcutaneous oxygen sensor 30 to the data storage unit 43.

The plurality of sensors 20 may further comprise an accelerometer 44 for detecting kinetic activity of the patient, and the accelerometer 44 may be connected with the data storage unit 43 so as to transfer and store output from the accelerometer 44 to the data storage unit 43. The data storage unit 43 may comprise one or more memory chip.

The diagnostic patch system 10 may further comprise a membrane button 46 configured to insert a marking event in the stream of the detected patient data. The diagnostic patch system 10 may further comprise an optical indicator 48 for indicating status of the sensors' contact to the skin surface. The optical indicator 48 may comprise one or more light-emitting diodes.

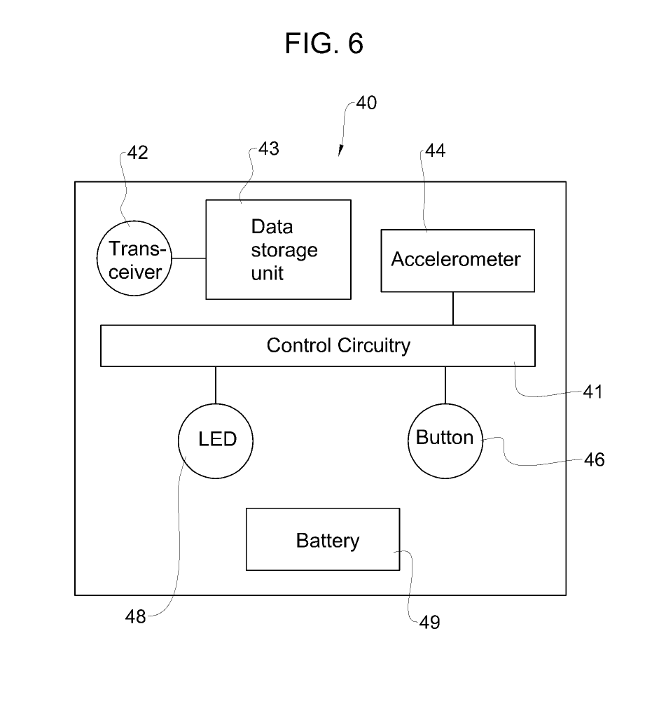

In an embodiment as shown in FIG. 6, the diagnostic patch system 10 may further comprise a circuitry board 40 comprising a control circuitry 41 configured to control behavior of the plurality of sensors 10, the data storage unit 43, and the transceiver 42. The transceiver 42 may be connected with the analyzer 90 through a wired communication or a wireless communication. Alternatively, the transceiver 20 may be connected with the analyzer 90 through a socket provided in the analyzer 90.

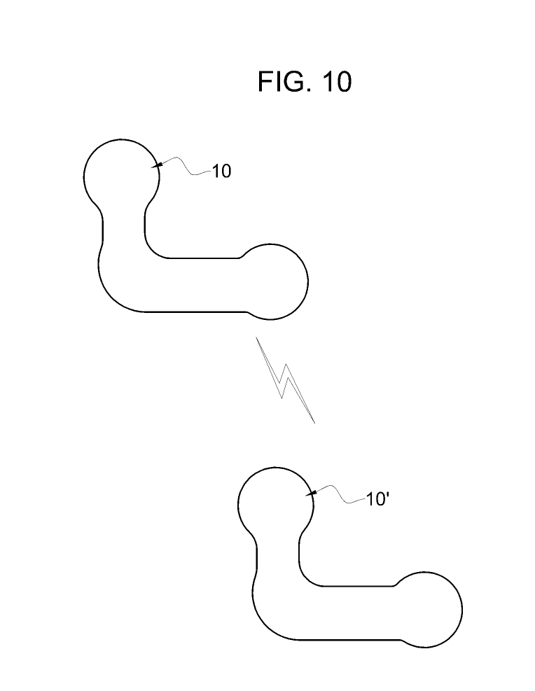

The analyzer 90 may comprise a computer. The diagnostic patch system 10 may be disposable. Alternatively, some of the components of the diagnostic patch system 10 may be reusable. The transceiver 42 may be configured to make a diagnostic patch system 10 communicate with another diagnostic patch system 10′ as shown in FIG. 10. The diagnostic patch system 10 may further comprise an electromotive force device 49 configured to provide electric power to the sensors 20, the data storage unit 43, and the transceiver 42. The electromotive force device 49 may comprise a battery.

The flexible support 60 comprises first and second adhesive surface portions 62, 64. The plurality of first adhesive surface portions 62 configured to engage one of the sensors 20, the data storage unit 43, and the transceiver 42. The plurality of second adhesive surface portions 64 configured to be attached to the skin of the patient. The plurality of second adhesive surface portions 64 comprise skin safe adhesive.

The sensors 20, the data storage unit 43, and the transceiver 42 are disposed apart from one another on the flexible support 60 so as to make the diagnostic patch system 10 substantially flexible across the disposed sensors 20, the data storage unit 43, and transceiver 42, and the diagnostic patch system 10 comprises a plurality of flexible electrical wires 70 connecting the sensors 20, the data storage unit 43, and the transceiver 42.

In a preferred mode, each of the first adhesive surface portions 62 may be substantially surrounded by the second adhesive surface portions 64 such that each of the sensors 20, the data storage unit 43, and the transceiver 42 is surrounded by the second adhesive surface portions 62 of the flexible support 60 and securely pressed down and contacted to the skin to obtain a good electrical contact.

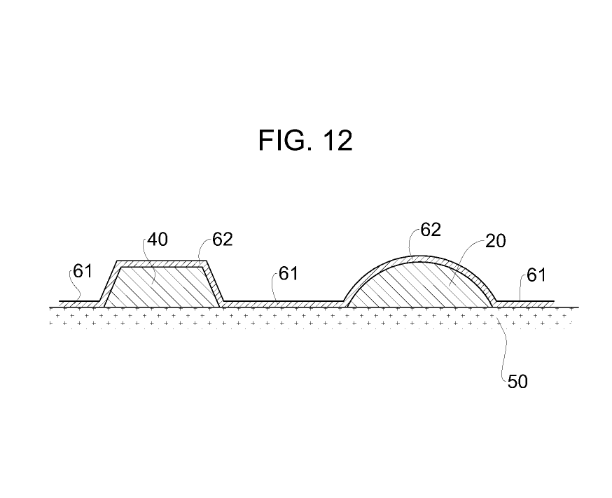

In another embodiment as shown in FIG. 12, the first adhesive surface portions 62 of the flexible support 60 are attached to tightly follow top surfaces 27 of the sensors 20, the data storage unit 43, and the transceiver 42 such that the second adhesive surface portions 64 are securely attached to the skin.

Each of the sensors 20, the data storage unit 43, and the transceiver 42 comprises a flat bottom surface 28 such that the first adhesive surface portions 62 meet the second adhesive surface portions 64 with angles larger than 90 degrees at borders between the first and second adhesive surface portions 62, 64 as seen in FIGS. 2 and 12, by which the sensors 20, the data storage unit 43, and the transceiver 42 are more securely attached to the skin to obtain more reliable and stable patient data therefrom.

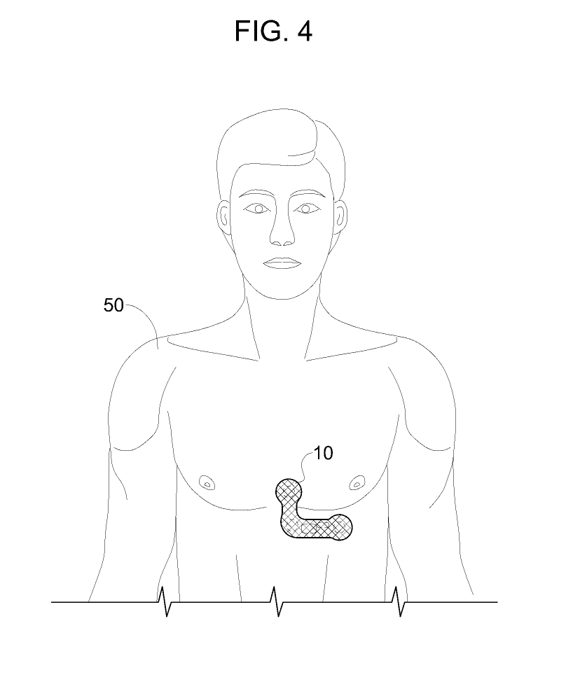

The diagnostic patch system 10 may further comprise a flexible support or patch substrate 60 configured to engage the plurality sensors 20, the data storage unit 43, and the transceiver 42 so as to fix them in place. The flexible support 60 may be further configured to attach the diagnostic patch system 10 to the skin of the patient 50 detachably as shown in FIG. 4.

Another aspect of the invention provides an analyzer 90 for processing and analyzing the stream of the patient data from a plurality of diagnostic patch systems 10 as shown in FIGS. 8 and 9. The analyzer 10 comprises an interface device 92, an information processing device 94, and one or more output devices 96. The interface device 92 is configured to communicate with each of the plurality of diagnostic patch systems 10.

The information processing device 94 is configured to analyze and interpret the stream so as to extract a plurality of temporal patient data and to correlate the plurality of temporal patient data with one another. The one or more output devices 96 are configured to present the temporal patient data.

The interface device 92 may comprise a socket having electrical terminals and a radio-frequency transceiver. The plurality of temporal patient data may comprise a plurality of ECG data, a temporal change of oxygen consumed by the patient, and a temporal change of kinetic variables. The plurality of temporal patient data may further comprise a cardiovascular data, and a pulmonary data.

While the invention has been shown and described with reference to different embodiments thereof, it will be appreciated by those skills in the art that variations in form, detail, compositions and operation may be made without departing from the spirit and scope of the invention as defined by the accompanying claims.

What is claimed is:

1. A diagnostic patch system, comprising:
a plurality of sensors attached to one or more skin surface areas to detect patient data;
a data storage unit to store therein stream of the patient data detected from the sensors;
a transceiver connected with the plurality of sensors and the data storage unit to communicate the stream of the patient data with an analyzer, wherein the analyzer is configured to process and analyze the stream of the patient data; and
a flexible support to engage thereto the sensors, the data storage unit, and the transceiver, wherein the support is detachably attached to one or more skin surface areas of a patient,
wherein the sensors comprise an electrocardiogram (ECG) sensor having skin surface electrodes, wherein the skin surface electrodes comprise two or more ECG channels, and wherein the at least two ECG channels are perpendicular to each other.

2. The diagnostic patch system of claim 1, wherein the data storage unit is configured to store the stream of patient data for about twenty four (24) to about forty eight (48) hours.

3. The diagnostic patch system of claim 2, wherein the diagnostic patch system is disposable after use.

4. The diagnostic patch system of claim 2, wherein the ECG sensor is connected with the data storage unit so as to transfer and store output from the ECG sensor to the data storage unit.

5. The diagnostic patch system of claim 1, wherein the sensors comprise a transcutaneous oxygen sensor, and wherein the transcutaneous oxygen sensor is connected with the data storage unit so as to transfer and store output from the transcutaneous oxygen sensor to the data storage unit.

6. The diagnostic patch system of claim 1, wherein the sensors comprise an accelerometer for detecting kinetic activity of the patient, and wherein the accelerometer is connected with the data storage unit so as to transfer and store output from the accelerometer to the data storage unit.

7. The diagnostic patch system of claim 1, wherein the data storage unit comprises one or more memory chips.

8. The diagnostic patch system of claim 1, further comprising a membrane button configured to insert a marking event in the stream of the detected patient data.

9. The diagnostic patch system of claim 1, further comprising an optical indicator for indicating status of the sensors′ contact to the skin surface.

10. The diagnostic patch system of claim 9, wherein the optical indicator comprises one or more light-emitting diodes.

11. The diagnostic patch system of claim 1, further comprising a control circuitry configured to control behavior of the sensors, the data storage unit, and the transceiver.

12. The diagnostic patch system of claim 11, wherein the transceiver communicates with the analyzer through a wired communication.

13. The diagnostic patch system of claim 11, wherein the transceiver communicates with the analyzer through a wireless communication.

14. The diagnostic patch system of claim 11, wherein the transceiver communicates with the analyzer through a socket provided in the analyzer.

15. The diagnostic patch system of claim 11, wherein the analyzer comprises a computer.

16. The diagnostic patch system of claim 1, wherein at least part of the diagnostic patch system is reusable.

17. The diagnostic patch system of claim 1, wherein the transceiver is configured to communicate with another diagnostic patch system.

18. The diagnostic patch system of claim 1, further comprising an electromotive force device configured to provide electric power to the sensors, the data storage unit, and the transceiver.

19. The diagnostic patch system of claim 18, wherein the electromotive force device comprises a battery.

20. The diagnostic patch system of claim 1, wherein the flexible support comprises:
a plurality of first adhesive surface portions configured to engage thereto one of the sensors, the data storage unit, and the transceiver, and
a plurality of second adhesive surface portions configured to be attached to the skin of the patient.

21. The diagnostic patch system of claim 20, wherein the plurality of second adhesive surface portions comprise a skin safe adhesive.

22. The diagnostic patch system of claim 1, wherein the sensors, the data storage unit, and the transceiver are disposed apart from one another on the flexible support so as to make the diagnostic patch system substantially flexible across the disposed sensors, the data storage unit, and the transceiver, wherein the diagnostic patch system comprises a plurality of flexible electrical wires connecting the sensors, the data storage unit, and the transceiver.

23. The diagnostic patch system of claim 22, wherein the flexible support comprises:
a plurality of first adhesive surface portions to engage thereto one of the sensors, the data storage unit, and the transceiver, and
a plurality of second adhesive surface portions configured to be attached to the skin of the patient,
wherein each of the first adhesive surface portions is substantially surrounded by the second adhesive surface portions such that each of the sensors, the data storage unit, and the transceiver is surrounded by the second adhesive surface of the flexible support and securely pressed down and contacted to the skin to obtain a good electrical contact.

24. The diagnostic patch system of claim 23, wherein the first adhesive surface portions of the flexible support are attached to tightly follow top surfaces of the sensors, the data storage unit, and the transceiver such that the second adhesive surface portions are securely attached to the skin.

25. The diagnostic patch system of claim 24, wherein each of the sensors, the data storage unit, and the transceiver comprises a flat bottom surface such that the first adhesive surface portions meet the second adhesive surface portions with angles larger than 90 degrees at borders between the first and second adhesive surface portions.

26. A diagnostic patch system, comprising:
a plurality of sensors attached to one or more skin surface areas to detect patient data representing cardiovascular activities of a patient;
a data storage unit to store therein stream of the patient data detected from the sensors;
a transceiver connected with the plurality of sensors and the data storage unit to communicate the stream of the patient data with an analyzer, wherein the analyzer is configured to process and analyze the stream of the patient data; and
a flexible support to engage thereto the sensors, the data storage unit, and the transceiver, wherein the support is detachably attached to one or more skin surface areas of the patient,
wherein the sensors comprise electrocardiogram (ECG) sensor having skin surface electrodes, wherein the skin surface electrodes comprise two or more ECG channels, and wherein the at least two ECG channels are perpendicular to each other.

27. The diagnostic patch system of claim 26, further comprising an interface unit between the diagnostic patch system and the analyzer.

28. The diagnostic patch system of claim 27, wherein the interface unit is configured to connect the diagnostic patch system and the analyzer through wireless communication.

29. The diagnostic patch system of claim 26, further comprising a membrane button configured to insert a marking event in the stream of the detected patient data.

30. The diagnostic patch system of claim 26, further comprising an optical indicator for indicating status of the sensors′ contact to the skin surface.

31. The diagnostic patch system of claim 30, wherein the optical indicator comprises one or more light-emitting diodes.

32. The diagnostic patch system of claim 26, further comprising a control circuitry configured to control behavior of the sensors, the data storage unit, and the transceiver.

33. An analyzer for processing and analyzing the stream of the patient data from a plurality of diagnostic patch systems of claim 1, the analyzer comprising:
an interface device configured to communicate with each of the plurality of diagnostic patch systems;
an information processing device configured to analyze and interpret the stream so as to extract a plurality of temporal patient data and to correlate the plurality of temporal patient data; and
one or more output devices configured to present the temporal patient data.

34. The analyzer of claim 31, wherein the interface device comprises an interface unit having electrical terminals and a radio-frequency transceiver.

35. The analyzer of claim 34, wherein the plurality of temporal patient data comprises a heart rate data, a plurality of ECG data, and a cardiovascular data.

36. A method for providing a plurality of billing information using a diagnostic patch system according to claim 1.

37. The method of claim 36, wherein the detected patient data stored in the data storage unit is processed by the analyzer to obtain the billing information for a plurality of insurance plans.

38. The method of claim 37, wherein the insurance plans comprise government insurance plans and private insurance plans.

39. The method of claim 37, wherein the billing information comprises a plurality of temporal data from the stream of the patient data.
